(19) United States
(12) Patent Application Publication
(10) Pub. No.: US 2022/0133231 A1
(43) Pub. Date: May 5, 2022

(54) WEARABLE DEVICES FOR DETECTING PHYSIOLOGICAL PARAMETERS

(71) Applicant: James R. Balman, Wichita, KS (US)

(72) Inventor: James R. Balman, Wichita, KS (US)

(21) Appl. No.: 17/429,225

(22) PCT Filed: Feb. 7, 2020
(86) PCT No.: PCT/US20/17308

§ 371 Date: Aug. 6, 2021

Related U.S. Application Data
(60) Provisional application No. 62/802,384, filed on Feb. 7, 2019.

(51) Int. Cl. A61B 5/00; A61B 5/145; A61B 5/024; A61B 5/1455

(52) U.S. Cl. CPC A61B 5/6806; A61B 5/14539; A61B 5/02411; A61B 2562/0257; A61B 5/0002; A61B 5/14546; A61B 5/14551

(57) ABSTRACT

A wearable device for detecting physiological parameters of a patient is provided. The device includes a flexible body for covering at least a portion a wearer's hand, the flexible body having an interior side and an exterior side. The device may also include a first sensor positioned on the body for measuring the pH of the patient on an exterior side of the flexible body. The device may further include a second sensor positioned on the body for measuring the bicarbonate level of the patient on an exterior side of the flexible body.

### CROSS-REFERENCE TO RELATED APPLICATIONS

This application claims benefit of U.S. Provisional Application No. 62/802,384, filed Feb. 7, 2019, which is hereby incorporated herein by reference in its entirety.

### FIELD OF THE INVENTION

This application generally relates to wearable devices to detecting physiological parameters of a patient and methods of use thereof.

### BACKGROUND OF THE INVENTION

Fetal acidosis is a high level of acid in the blood of a fetus resulting from a limited oxygen supply available to the fetus over an extended period of time. Known tests for fetal acidosis, such as Doppler Ultrasonography, fetal heart rate monitoring, physical examination, and fetal blood tests are invasive or have unacceptable margins of error. For example, cordocentesis, an ultrasound-guided procedure to collect fetal blood from the umbilical cord, may not be used for routine or repeated monitoring due to its procedure-related risk. Additionally, fetal scalp sampling, an operation where the fetus's head is pierced to obtain the level of pH in the tissue, is another invasive, unreliable procedure used to attempt to diagnose fetal acidosis.

A need exists for a procedure that is less invasive to the fetus but which allows medical professionals to reliably determine whether a fetus is experiencing fetal acidosis while a patient is in labor. Similarly, need exists for the ability to quickly and easily monitor fetal asphyxia.

Further, when interacting with a patient, in some cases a medical professional's hands may not be free to obtain or manipulate multiple devices at the same time. For example, the medical professional's hands may be in a compromised position and unable to reach for or hold a probe. Even in instances when the medical professional can interact with a probe, it may not be easy or convenient for the professional to determine where the probe may be contacting. For example, when evaluating a pregnant person and/or a fetus, the professional may be using his or her hands to inspect the patient. Oftentimes, the medical professional can determine where and what type of tissue that is being engaged. It would be helpful if the medical professional could quickly determine physiological parameters for the patient without the medical professional having to remove his or her hands.

Similarly, edema build up can distort many sensors readings when evaluating a pregnant person and/or fetus. It would be helpful if a medical professional could use his or her fingers to feel locations of non-edema build up to provide more accurate readings.

In other forms, pulse oximetry may be used to evaluate patients. However, such readings can be especially difficult to obtain for a fetus. For example, it can be very difficult to determine if the sensor is contacting the pregnant person, the fetus, or intermediate tissue. Further, transmissive oximetry can be more accurate than reflective oximetry, but it is especially difficult to determine a proper location for transmissive oximetry for a fetus. It would be helpful if a device or system were available to help guide a user to an appropriate location and also determine if transmissive oximetry would be appropriate by having the sensor components near enough to one another.

### SUMMARY OF THE INVENTION

Wearable devices allow sensors to the be positioned outward on the finger tips and finger sides of the person wearing the assembly. In one form, the sensors are pointed outward and when in proximity of a patient, such as a fetus, can provide health status data. In one form, these sensors provide health status data of the fetus that can be interpreted by a processor connected wireless and/or in a wired manner. In one form, to prevent false readings, the wearable device takes pulse readings from the wearer to prevent accidentally reading the wearer's vitals. The pulse readings from the wearer and pregnant person are compared to the sensors located on the fingers determining if the finger sensors have incorrect data. The input and output data can store locally, remotely, and/or transmitted via radio waves.

From the pH sensor's contact with the fetus and a subsequent pH reading which may correlate to the pH of the fetus's blood, the user may reliably determine whether a fetus is experiencing fetal acidosis and take appropriate countermeasures. Other physiological parameters which may also help support a diagnosis fetal acidosis in-utero are pulse rate of the fetus and/or an oxygen saturation level of the fetus's blood.

The apparatus may also include a pressure sensor. In some embodiments, the pressure sensor may be a pressure switch. The apparatus may also include an optical sensor. In one form, the optical sensor may be a pulse oximeter, which may allow for a user to obtain the pulse rate reading of a surface that is contacted with the pH sensor. This pulse rate reading of the surface contacted may be compared to an external reading of a pulse rate of the patient to confirm whether the pH sensor is contacting the fetus or the patient. If the pulse rates are similar, the pH sensor is contacting the patient. If the pulse rates are dissimilar, then the pH sensor is contacting the fetus.

In one form, a wearable device for detecting physiological parameters of a patient is provided. The device includes a flexible body for covering at least a portion a wearer's hand, the flexible body having an interior side and an exterior side. The device may also include a first sensor positioned on the body for measuring the pH of the patient on an exterior side of the flexible body. The device may further include a second sensor positioned on the body for measuring the bicarbonate level of the patient on an exterior side of the flexible body.

In accordance with one form, a wearable device may be provided including a flexible body, a first sensor component, a second sensor component, and a proximity sensor. The flexible body for covers at least a portion a wearer's hand and has an interior side and an exterior side. The second sensor component is located remotely from the first sensor component. The proximity sensor is adjacent at least one of the first and second sensor components. At least one of the first and second sensor components is configured for use as a reflective oximetry sensor and a transmissive oximetry sensor for the patient on the exterior side of the flexible body. The at least one of the first and second sensor components is configured to permit transmissive sensing when the proximity sensor detects the first and second sensor components are within a specified distance from one another and permit reflective sensing when the proximity sensor detects the first and second sensor components are beyond a specified distance from one another.

In one form, a system for detecting physiological parameters of a patient is provided. The system includes a first body, a second body, a first sensor, a second sensor, and a display. The first body covers at least a portion of one of a wearer's fingers with the first body having an exterior side and an interior side. The second body covers at least a portion of another of the wearer's fingers with the second body having an interior side and an exterior side. The first sensor is positioned on the first body. The first sensor is configured to detect at least one of pH, pulse, oxygen saturation, bicarbonate levels, and cranial pressure of the patient on an exterior side of the first body. The second sensor is positioned on the second body. The second sensor is configured to detect at least one of pH, pulse, oxygen saturation, bicarbonate levels, and cranial pressure of the patient on an exterior side of the first body. The display is positioned remotely from the first and second bodies. The display provides information indicative of sensed values from the first and second sensors.

In accordance with one form, a wearable device is provided for detecting physiological parameters of a patient. The device includes a flexible body, a first sensor, and a second sensor. The flexible body covers at least a portion a wearer's hand and has an interior side and an exterior side. The first sensor is positioned on the body for measuring the pH of the patient on an exterior side of the flexible body. The second sensor is positioned on the body. The second sensor determines at least one of the pulse and oxygen saturation of the patient on the exterior side of the flexible body.

In accordance with one form, the body is in the form of a glove with a palm portion that is unitary with finger portions. The first and second sensors are positioned on at least one finger portion.

According to one form, the first sensor includes a central pH sensor portion and an outer support portion, the outer support portion having a generally curved or sloped shape.

In one form, the device further includes a processor and a display.

In accordance with one form, the device further includes a conductive ink extending along at least a portion of a connection path between at least one of the first and second sensors and at least one of the processor and display.

According to one form, the device further includes a pulse oximeter sensor.

In one form, the device further includes a proximity sensor.

In accordance with one form, the device further includes a pressure switch.

According to one form, the device further includes a photo sensor.

In one form, the device further includes a plurality of at least one of the first and second sensors located on different portions of the flexible body.

In accordance with one form, the device further includes a reference sensor for measuring a physiological parameter of the wearer on the interior side of the flexible body.

According to one form, thee device further includes a wireless transmitter.

In one form, the first and second sensors are formed as a single sensor array at a single location on the flexible body.

### BRIEF DESCRIPTION OF THE DRAWINGS

FIG. 1 is a perspective view of one form of a wearable device in the form of a glove;

FIG. 2A is a top view of one form of a wearable device in the form of a glove;

FIG. 2B is a bottom view of the wearable device of FIG. 2A;

FIG. 3 is a top view of one form of a sensor array;

FIG. 4 is a bottom view of a wearable device showing various locations of sensor positions;

FIG. 5 is a bottom view of multiple wearable devices as part of a system;

FIG. 6A is a side view of one form of a pH sensor;

FIG. 6B is a side view of another form of a pH sensor;

FIG. 7 is a process flow diagram of one form of determining physiological parameters;

FIG. 8 is a perspective view of another form of a system for detecting physiological parameters;

FIG. 9A is a perspective view showing one use of a wearable device;

FIG. 9B is a perspective view showing another use of a wearable device;

FIG. 9C is a perspective view showing another use of a wearable device;

FIG. 10 is a diagrammatic view showing another use of a wearable device; and

FIG. 11 is a diagrammatic view showing wireless communication between a wearable device and a remote display.

### DETAILED DESCRIPTION

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

As described below, a variety of wearable devices and associated systems are described. It should be appreciated that such devices include gloves, finger covering thimble-like portions, and the like. While various embodiments may be shown as being in the form of such devices, it should be understood that the sensors, connections, circuitry, etc. can be implemented in any of the forms described herein. For example, while a configuration of certain sensors may be shown on a glove form, it should be understood that those sensors and connections may also be implemented in a thimble-like device covering just the fingers of the wearer.

Additionally, it should be appreciated that a variety of sensors are also described herein. While certain sensors may be described as being positioned on any one of the devices and locations on the devices, it should be appreciated that the sensors may be moved to other locations on the devices. Similarly, while a specific type of device may be illustrated as being positioned on a certain portion of the device, it should be understood that other types of sensors may be also included or included in the alternative.

Many of the embodiments described herein may reference the birthing process, pregnant persons, a fetus, and the like. However, it should be appreciated that the devices and systems described herein may be used for other medical purposes, such as on a non-pregnant person, on animals, and the like.

Cervical checks are generally part of the birthing care process. With a fingertip sensor made specifically for detecting physiological parameters of one or more of the pregnant person and the fetus, one can obtain vital data while performing a cervical check without additional vaginal probing. Further, as noted above, edema build up can distort many sensors readings. Using fingers to feel locations of non-edema build up can provide care takers more accurate readings. In general, fingertip sensors allow for more accurate sensor placement & more accurate reading.

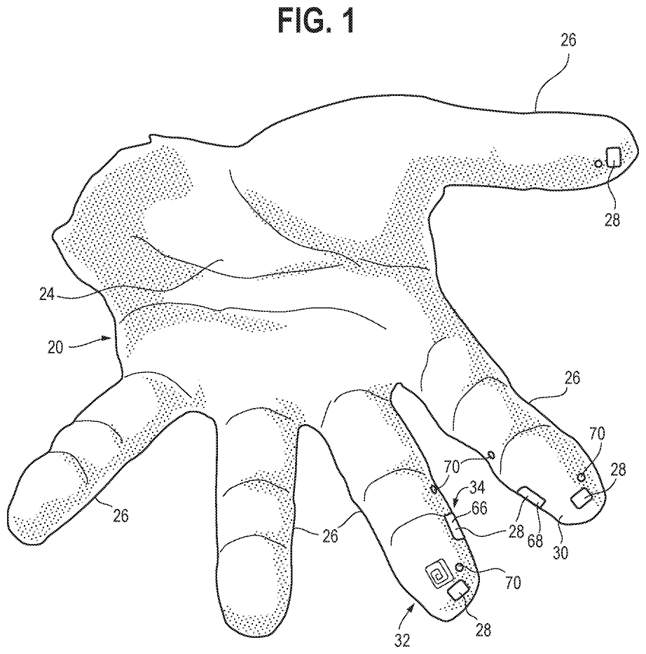

As shown in FIG. 1, a wearable device 20 is shown in the form of a glove. The wearable device includes a body 22 that can be made from a variety of materials. For example, such materials can include flexible materials such as latex, nitrile rubber, polyvinyl chloride, neoprene, low density polyethylene, and the like.

In one form, the body 22 includes a palm portion 24 and finger covering portions 26. In one form, the body 22 is generally integral such that the palm portions 24 and finger covering portions form a single unit, such as a standard medical glove. The body may also have a flexible membrane over at least some of the structures, wiring, and the like described in more detail below.

As shown in FIG. 1, the device 20 includes a plurality of sensors 28 located at various locations on the body 22. Not all of the sensors 28 are specifically identified in FIG. 1 and some are referred to as more specific forms of sensors, as described below. However, it should be appreciated that the descriptions provided herein may be applicable to all sensors shown and described herein. The sensors 28 are suitable for detecting various physiological parameters and/or information usable to provide guidance regarding a physiological parameter. As will be described in more detail below, such sensors 28 include, but are not limited to pH sensors, pulse oximeters, bicarbonate sensors, pressure sensors, reference sensors, proximity sensors, and the like.

The sensors 28 can be configured such that the are exposed on an exterior surface 28 of the body 22. In other forms, the sensors are positioned on an interior side of the body 22, but still are configured to obtain physiological parameters of a patient on an exterior side 30 of the body 22. As will be described below, the device 20 may also include a reference sensor positioned on an interior side to measure at least one physiological parameter of the wearer.

The sensors 28 can be positioned at various locations on the body 22. For example, as shown in FIG. 1, the sensors can be positioned on a finger tip portion 32, an inside portion 34 of the finger, as well as other locations. Furthermore, multiple sensors can be positioned at various locations. In one form, multiple forms of the same type of sensor 28 can be positioned at different locations. For example, a pH sensor can be positioned on a finger tip portion as well as an inside of the finger portion. The location of the sensors 28 allows more accurate readings based on the articulation of the fingers while in limited movement environments. Further, multiple sensor locations provide multiple feedback opportunities. This becomes helpful in environments where the optimal sensor location cannot be achieved with one static sensor location.

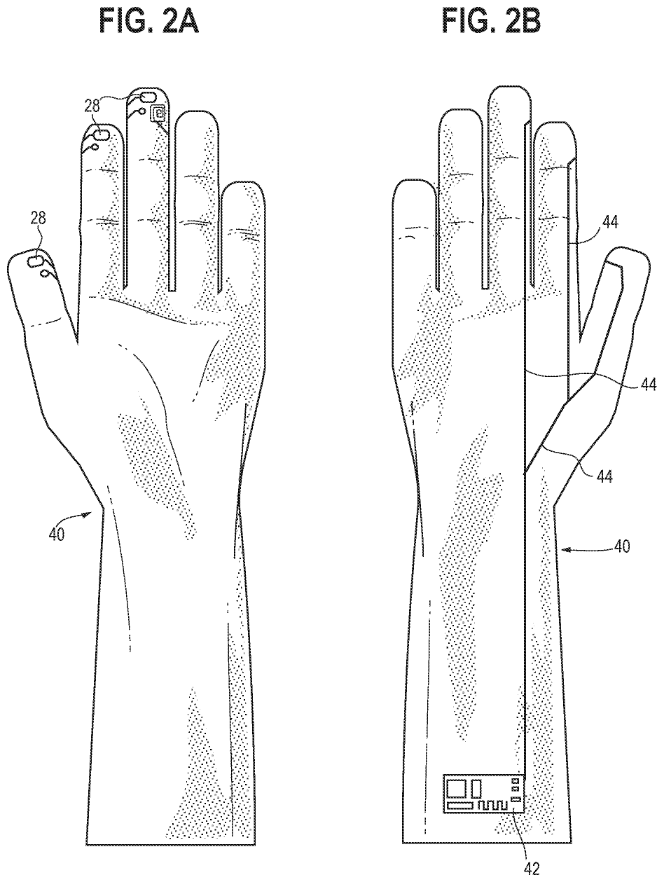

Referring now to FIGS. 2A and 2B, another form of a wearable device 40 is shown. Device 40 can include similar features as described above. The views shown in FIGS. 2A and 2B further illustrate that the device can include a processor 42 that is coupled to sensors 28. The processor 42 can be coupled to the sensors via wired connections 44. The wired connections 44 can take a variety of forms, such as standard wires, conductive ink, printed connections, and the like. In some forms, the device can be formed whereby one layer is a dielectric stretchable membrane with a printable conducting ink (silver, carbon, etc.) thereon with an additional encapsulation layer covering the printed ink or wiring layer. In some forms, the sensors 28 may be wirelessly coupled to the processor 42, such as using radio waves and the like.

The processor 42 can be used to interpret the information provided by the sensors 28, such as determining and outputting the pH, oxygen saturation, bicarbonate levels, pulse, and the like. In some forms, the processor 42 can include a display and/or may connect to a display located elsewhere. The processor may also form part of microcircuitry having a wireless transmitter for communicating with external devices, such as monitors, medical recording equipment, and the like.

In some embodiments, the processor may be operatively coupled to a memory, which may be configured to store the information received from the sensors for a patient for a certain period of time. In some embodiments, the device may include circuitry for communication with specific applications or devices such as an external or existing monitoring device's screen which receives information from the processor to alert a user to changes in the pH and temperature of the fetus's blood, heart rate variability of the fetus, or other physiological parameters. The change in pH of the fetus's blood may be indicative of complications in oxygen delivery and may lead to further problems, such as fetal acidosis. The user may receive information regarding the physiological changes from a mobile electronic device that operates an application and receives information from the processor.

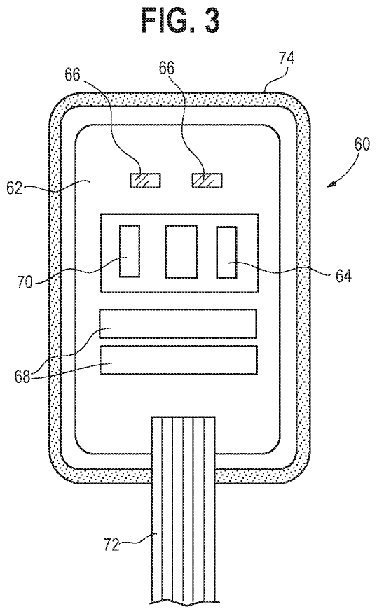

A plurality of sensors and/or sensor components can be assembled together as a sensor array 60, as shown in FIG. 3. In this form, a variety of different sensors can be assembled together into a single unit. For example, as shown in FIG. 3, a pressure sensor 62, a pH sensor 64, a pulse oximeter having IR and/or LEDs 66 and photodiodes 68, and a proximity sensor 70 can be included in a single assembly. It should be appreciated that other combinations of sensors can be included in a single assembly and can also include other sensors such as a bicarbonate sensor. Similar to the features described above, the array can be wired or wirelessly connected to a processor and display. As shown in FIG. 3, a flexible wired connection 72 can be used. In some forms, the processor and/or communications circuitry may be built into the array. The array 60 can also include a flexible housing 74.

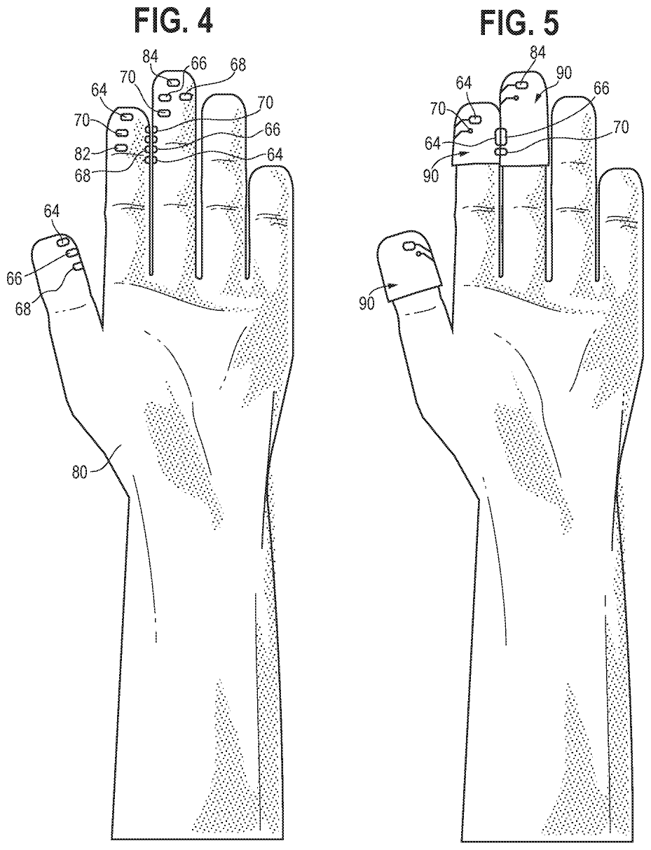

Further embodiments are shown in FIGS. 4 and 5. The embodiment shown in FIG. 4 more specifically identifies various positions of sensors on one form of a device. As noted above, the location, number, and types of sensors can be modified as desired. Device 80 includes pH sensors 64, IR or LEDs 66 and photodiodes 68 for a pulse oximeter, proximity sensors 70, pressure sensors 62, and bicarbonate sensor 84. As shown in FIG. 4, a variety of different sensors can be placed at various locations on the device 80, with multiple examples of each type of sensor so that different parts of the wearer's hand may contact the desired portion of the patient when in use.

It should also be appreciated that a reference sensor 82 may be included in any of the devices described herein, such as shown in FIG. 4. The reference sensor 82 may be configured to measure a physiological parameter of the wearer. The parameter may be pulse rate or any other parameter. This information from the reference sensor 82 can then be compared against information from other sensors detecting on an exterior of the device. The reference can then be used to help determine if the measurements are being taken from the wearer, the patient, or a different source. In the case of a pregnant person and fetus, the reference can be used to compare the wearer, the pregnant person (via another reference sensor), and the fetus.

Another form of device is shown in FIG. 5, such as in the form of thimble-like cover devices 90. The devices 90 can function independently and/or cooperate with and communicate with one another or otherwise form part of a system. The devices 90 can include any of the sensors and combinations thereof, as discussed above. The devices 90 can each include processors, communication devices, and the like or may be coupled to one another or to a processor and/or display. In one form, the devices 90 wirelessly communicate with one another and/or a central processor. Though not shown, the thimble-like devices may also include a tether or other supports to secure the devices to the wearer. For example, a flexible membrane can be connected to one or more of the devices and then extend to the wearer's wrist or otherwise connect to a display worn by the wearer.

Various aspects of different sensors will now be discussed in more detail. As noted above, a variety of different types of sensors can be used with the wearable devices described herein. One such sensor is a pH sensor. In some forms, the pH sensor includes a temperature sensor either built in or separate. The temperature sensor can be used to help maintain an accurate reading for the pH sensor. Various types of pH sensors can be used including, but not limited to ISFET pH (ion-sensitive field-effect transistor) sensors and the like. Prior sensors were typically built with glass or other materials that were not generally appropriate for the small size necessary for use with a wearable device. Further, pH sensors made with glass would be prone to sensor drift, thereby decreasing accuracy. The fabrication of ISFET sensors using new methods including, but not limited to, light curable materials allows the small size and accuracy.

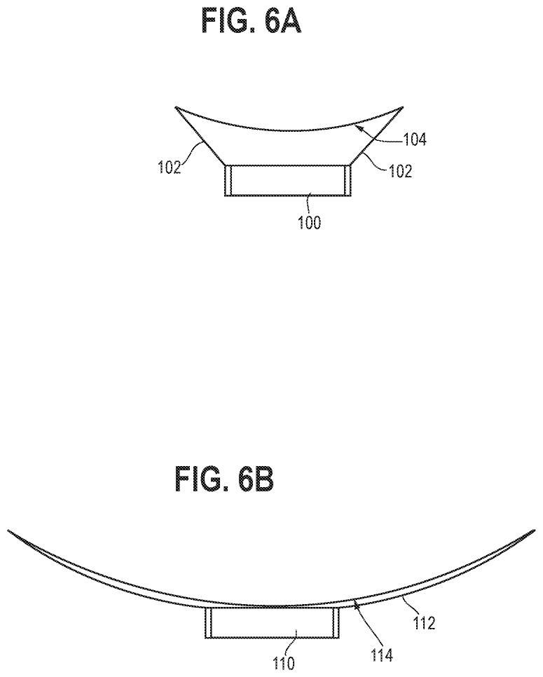

The assembly of the ISFET pH sensors may impact the performance in the devices described herein. In one form, the sensor mount must have a subtle approach leading to the sensor allowing skin contact. The sensors must have surrounds that protect the sensor from various environmental factors and premature contact with other surfaces. Most small ISFET sensors are not built in a way that allows skin contact. As shown in FIG. 6A, pH sensor 100 includes side walls 102 that are near the sensor 100 and have a sharp approach. The sharp approach of the sidewalls 102 generally prevents skin 104 from contacting the sensor 100. This can lead to inaccurate readings and permit intervening fluid to give false readings.

In another form, such as shown in FIG. 6B, a gradual taper to the sensor mount may provide for a better contact with skin, Sensor 110 includes a surrounding area, such as sidewalls 112 that taper gradually, thereby permitting skin 114 to contact the sensor 110.

Additionally, certain pH sensors, such as ISFET sensors are susceptible to light interfering with detection and accuracy. In this regard, other sensors, such as pulse oximetry, sensors that emit various forms of light, can interfere with the pH sensors. Therefore, in some forms, the devices can be configured that the pH sensor and pulse oximetry emitter/sensor cycle such that they are not detecting at the same points in time. This can also be enhanced by using proximity sensors to detect when the pulse oximetry emitter and sensor are near one another for operation. (? Anything else unique or interesting about the pH sensor, configuration of the pH sensor, use in the environment, etc.?)

As noted above, the pulse oximetry sensor can be a single sensor and/or comprise separate components. In some forms, the pulse oximetry sensor can include an emitter, such as LEDs or IR emitters, and detectors, such as photo diodes. These components can be assembled into a single sensor unit, such as shown in FIG. 3, and/or can be used as separate components located at different portions of the device, such as shown in FIGS. 4 and 5.

There are two main types of pulse oximetry sensors, reflective and transmissive. Reflective sensors emit and receive from generally the same surface relative to the patient. Transmissive, on the other hand, will emit on one side of the patient's tissue and receive on the other. The devices herein can include both types of pulse oximetry sensors. The emitter may, include Light Emitting Diodes (LEDs) that emit light of different peak emission wavelengths, including, but not limited to, infrared light. The light may pass through the fetus's skin may be reflected off the fetus's subcutaneous bone and tissue before being received by the photodetector. The change in absorbance of the light emitted at each wavelength may be correlated to the level of oxygen saturation in the fetus's blood. The rate of change of the absorbance may be correlated to the observed pulse rate, which may be used to confirm contact with the fetus. For example, if the observed pulse rate is high as compared to the external pulse rate of the pregnant person, then contact with the fetus may be confirmed. For example, during labor, a pregnant person may have a pulse rate of 118 beats per minute, and the observed pulse rate may be 145 beats per minute, which may confirm contact with the fetus.

Further, the device can determine if the sensors are near one another and/or in contact with the patient, to then switch to transmissive sensing. Further, as noted above, in some forms, the pulse oximetry needs to cycle relative to pH sensing so that the light emitted from the pulse oximeter does not interfere with the pH sensing. Other sensors using refraction detection of one or more spectrums of light mays also need to be cycled so the light does not interfere with other sensors, such as the pH sensor.

The sensors may also include a bicarbonate sensor. The bicarbonate sensor may take a variety of forms, such as a bicarbonate ISFET sensor. The sensor can be augmented by modifying the filer for ion exchange. Further,

pH of the blood is a well-known metric for determining acidosis or lack of oxygen. The metabolic process of an oxygen deprived body will create acid. The increased acid causes the blood pH to change. To prevent cellular damage the body maintains bicarbonate as a buffer to mitigate cellular damage and or death. To identify bicarbonate levels with the combination of pH allows a practitioner to identify the condition of the blood status and if it has the ability to recover.

As noted above, the sensors may also include one or more proximity sensors. The proximity sensors can be used to determine if one or more of the other sensors are near one another. For example, for the pulse oximetry sensors, if the emitter and detector are close to one another such that transmissive sensing can be used. The proximity sensor can also be used in combination with other sensors, such as by determining if a pH sensor is near a light emitter.

Pressure switches/sensors can also be used in the devices and systems described herein. Pressure switches and sensors can be used to determine when the device is actually in contact with tissue and thereby activate the other sensors on the device. This can help prevent premature readings from being recorded. In one form, the pressure sensors can be used to activate one or more sensors. This can automate sensor activation, can save battery life, and can help avoid the wearer from having to activate the sensors.

Another sensor that can be used is a photo sensor. In one form, the photo sensor can be used to determine skin pigmentation that is exterior to the device, which can be used to help calibrate the oximetry sensors. The photo sensors can also be used to transmit images. In other embodiments, a laparoscopic light and/or camera may provide the user a visual indication to the user when the device has contacted a surface of the fetus. In one form, the photo sensors can be built into the oximetry sensors to create spectrums to light including both visible and infrared.

As noted above, the device may also include a reference sensor. The reference sensor can detect a physiological parameter of the wearer on an interior side of the device to compare with sensors detecting on n exterior side of the device. This can be used to determine if sensed information is from the desired source instead of the wearer. The reference sensor can be placed anywhere in the internal structure and in contact with the skin of the wearer. Similarly, an external reference sensor can be coupled to the device or system. The external reference sensor can be sensing information from the pregnant person to compare against data from the device.

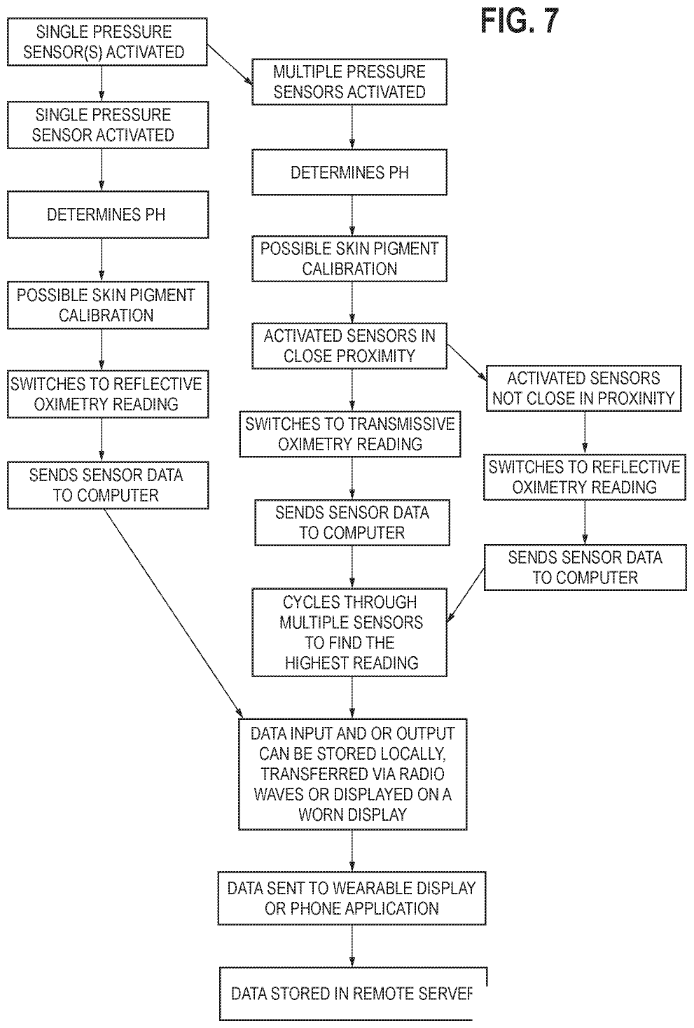

One exemplary method of sensing physiological parameters is shown in FIG. 7. As shown in this figure, the device and/or system can be activated by a pressure switch to then start taking pH readings. The device can also perform a skin pigment analysis, such as from a photo sensor, to calibrate the pH readings. Further the method can determine if reflective or transmissive oximetry readings should be taken.

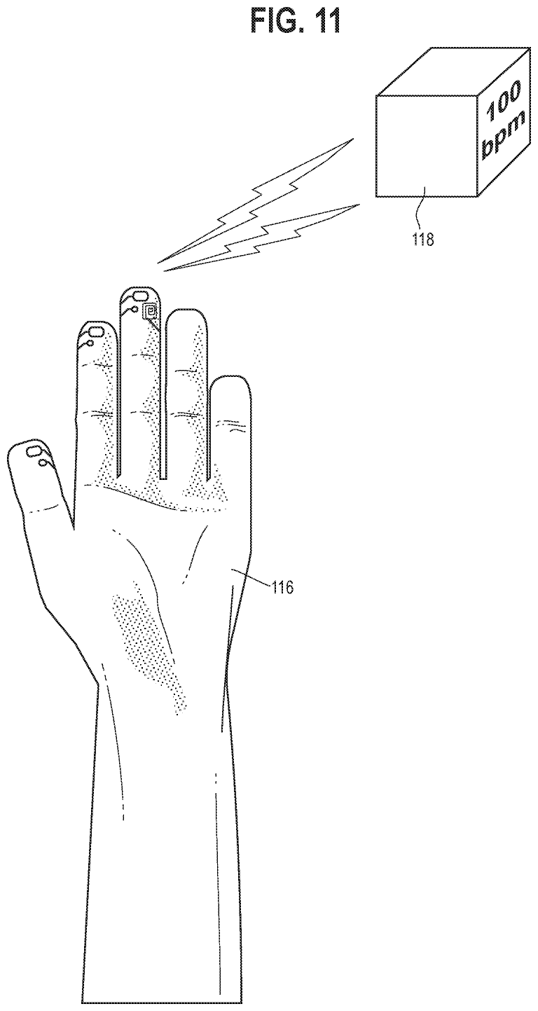

As described in FIG. 7, the resulting data can be displayed, stored, etc. locally or remote from the device or system. FIG. 11 presents a diagrammatic representation of a device 116 communicating wirelessly with a one or more of a processor, storage location, and display. In one form, display 118 can present information from the device 116.

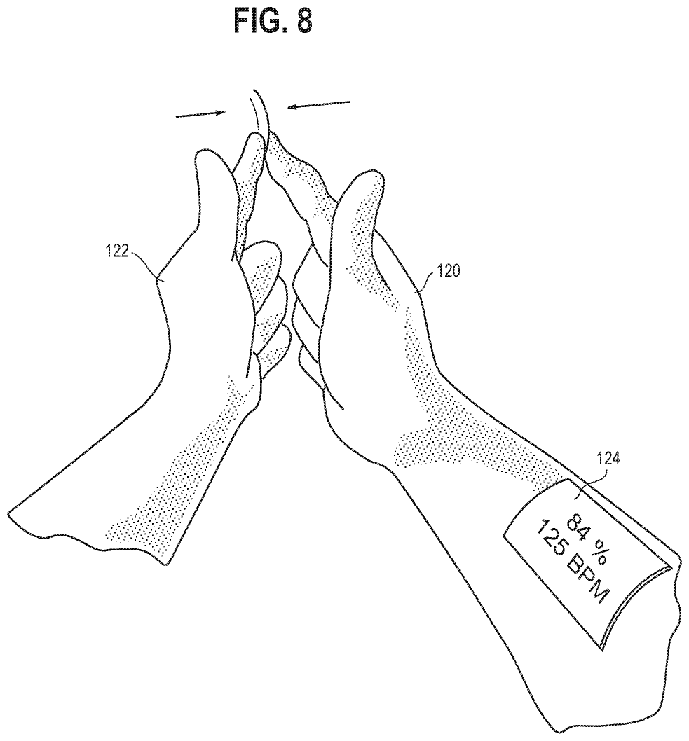

Another form of a wearable device is shown in FIG. 8, As show in this form, multiple devices 120,122 cooperate with one another. Sensors on the finger portions of each of the devices 120,122 can be used in instances where it is appropriate to use two hands. For instance, an emitter for a pulse oximeter on one device 120 can be used in combination with a detector on the other device 122 when proximity sensors indicate the devices 120,122 are within a specific distance of one another. The information from either or both of the devices 120,12 can be output on a display 124 that can be positioned on the forearm of the wearer. This can permit quick viewing of the information from the devices. It should be understood that similar displays may be used with any of the devices described herein.

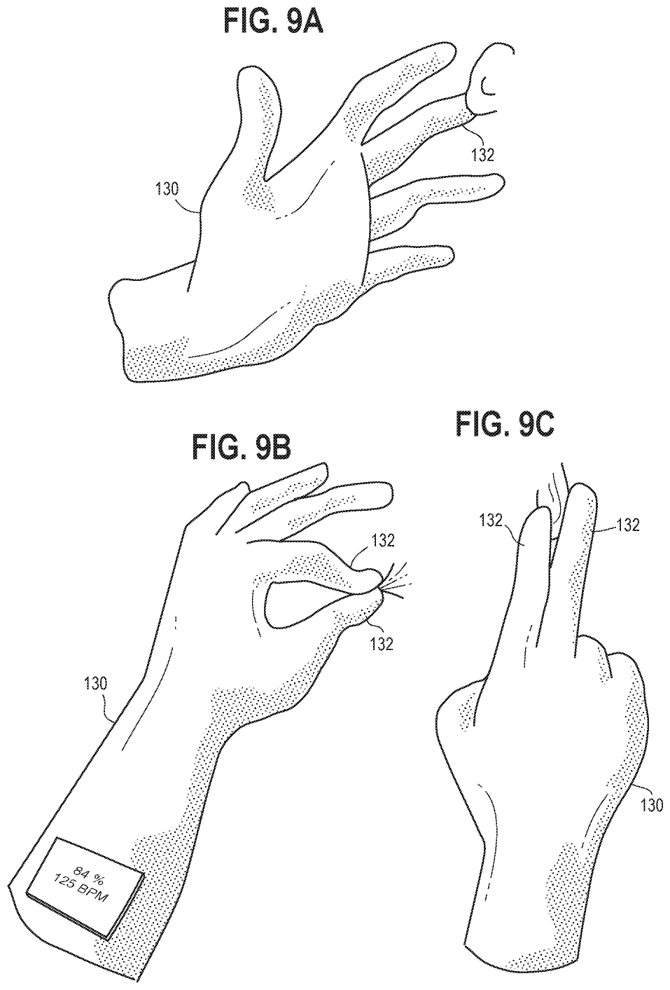

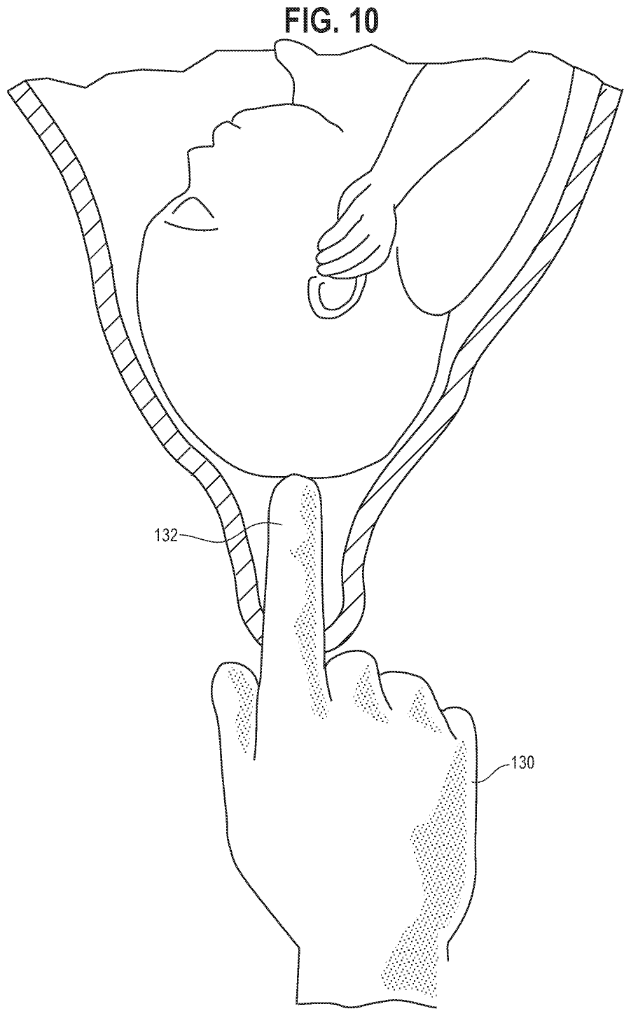

FIGS. 9A-9C illustrate various uses of device 130 having the features described herein. As shown in FIG. 9A, a single finger portion 132 having sensors thereon (not shown) can be used to detect and measure physiological parameters on a portion of a patient. FIG. 9B illustrates how two finger portions 132 can be used to pinch and detect physiological parameters between the two finger portions. For instance, an emitter on one finger portion can emit a signal that is detected on the other finger portion. Similarly, FIG. 9C illustrates how sensors on inside portions of the finger portions can be used in a similar manner as shown in FIG. 9B,

FIG. 10 illustrates yet another example whereby device 130 can be used to detect physiological parameters of a fetus, in-utero. By placing different types of sensors and multiple versions of the same sensors at various locations on the device, different configurations can be used on a patient. This can be especially helpful for detecting physiological parameters of a fetus where it may be difficult to contact a specific portion of the device with a specific portion of the fetus.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of Applicant's contribution. The actual scope of the protection sought is intended to be defined in the following claims when viewed in their proper perspective based on the prior art.

1. A wearable device for detecting physiological parameters of a patient comprising:
a flexible body for covering at least a portion a wearer's hand, the flexible body having an interior side and an exterior side;
a first sensor positioned on the body for measuring the pH of the patient on an exterior side of the flexible body; and
a second sensor positioned on the body for measuring the bicarbonate level of the patient on an exterior side of the flexible body.

2. The wearable device of claim 1 wherein the body is in the form of a glove with a palm portion that is unitary with finger portions, the first and second sensors being positioned on at least one finger portion.

3. The wearable device of claim 1 wherein the first sensor includes a central pH sensor portion and an outer support portion, the outer support portion having a generally curved or sloped shape.

4. The wearable device of claim 1 further comprising a processor and a display.

5. The wearable device of claim 4 further comprising a conductive ink extending along at least a portion of a connection path between at least one of the fast and second sensors and at least one of the processor and display.

6. The wearable device of claim 1 further comprising a pulse oximeter sensor.

7. The wearable device of claim 1 further comprising a proximity sensor.

8. The wearable device of claim 1 further comprising a pressure switch.

9. The wearable device of claim 1 further comprising a photo sensor.

10. The wearable device of claim 1 further comprising a plurality of at least one of the first and second sensors located on different portions of the flexible body.

11. The wearable device of claim 1 further comprising a reference sensor for measuring a physiological parameter of the wearer on the interior side of the flexible body.

12. The wearable device of claim 1 further comprising a wireless transmitter.

13. The wearable device of claim 1 wherein the first and second sensors are formed as a single sensor array at a single location on the flexible body.

14. A wearable device for detecting physiological parameters of a patient comprising:
a flexible body for covering at least a portion a wearer's hand, the flexible body having an interior side and an exterior side;
a first sensor component;
a second sensor component located remotely from the first sensor component; and
a proximity sensor adjacent at least one of the first and second sensor components,
at least one of the first and second sensor components configured for use as a reflective oximetry sensor and a transmissive oximetry sensor for the patient on the exterior side of the flexible body, the at least one of the first and second sensor components configured to permit transmissive sensing when the proximity sensor detects the first and second sensor components are within a specified distance from one another and permit reflective sensing when the proximity sensor detects the first and second sensor components are beyond a specified distance from one another.

15. The wearable device of claim 14 wherein the body is in the form of a glove with a palm portion that is unitary with finger portions, the first and second sensors being positioned on at least one finger portion.

16. The wearable device of claim 14 further comprising a pH sensor which includes a central pH sensor portion and an outer support portion, the outer support portion having a generally curved or sloped shape.

17. The wearable device of claim 14 further comprising a processor and a display.

18. The wearable device of claim 17 further comprising a conductive ink extending along at least a portion of a connection path between at least one of the first and second sensors and at least one of the processor and display.

19. The wearable device of claim 14 further comprising a pressure switch.

20-23. (canceled)

24. A system for detecting physiological parameters of a patient comprising:
a first body covering at least a portion of one of a wearer's fingers, the first body having an exterior side and an interior side;
a second body covering at least a portion of another of the wearer's fingers, the second body having an interior side and an exterior side;
a first sensor positioned on the first body, the first sensor configured to detect at least one of pH, pulse, oxygen saturation, bicarbonate levels, and cranial pressure of the patient on an exterior side of the first body;
a second sensor positioned on the second body, the second sensor configured to detect at least one of pH, pulse, oxygen saturation, bicarbonate levels, and cranial pressure of the patient on an exterior side of the first body; and
a display positioned remotely from the first and second bodies, the display providing information indicative of sensed values from the first and second sensors.

25-48. (canceled)
